(19) United States
(12) Patent Application Publication
(10) Pub. No.: US 2003/0061629 A1
(43) Pub. Date: Mar. 27, 2003

(54) Production of transgenic birds using stage X primordial germ cells

(76) Inventor: Pramod Sutrave, Athens, GA (US)

Correspondence Address: PENNIE & EDMONDS LLP, 1155 AVENUE OF THE AMERICAS, NEW YORK, NY, 10036-2711, US

(21) Appl. No.: 09/992,634

(22) Filed: Nov. 14, 2001

Related U.S. Application Data
(60) Provisional application No. 60/324,014, filed on Sep. 21, 2001.

(51) Int. Cl. A01K 67/027; C12N 5/06

(52) U.S. Cl. 800/19; 435/349

(57) ABSTRACT

The present invention relates to methods for isolating primordial germ cells (PGCs) from the blastoderm of a stage X avian embryo. The present invention further relates to methods for producing a transgenic bird by modifying the isolated PGCs, such that the cells incorporate at least one transgene into their genetic material; transferring the modified PGCs into a suitable recipient, such as a blastoderm of an avian embryo, hatching the embryo; and testing for the presence of the transgene or expression of the protein encoded by the transgene.

The present application claims the benefit of priority from a provisional application filed Sep. 21, 2001 and having U.S. Serial No. 60/324,014.

### FIELD OF THE INVENTION

The invention relates to methods for isolating stage X primordial germ cells from the blastoderm of an avian egg. The present invention further relates to methods for generating transgenic avians by transfecting isolated stage X primordial germ cells that are then implanted in an avian blastoderm to develop into a mature transgenic bird.

### BACKGROUND

The field of animal transgenics was initially developed to understand the action of a single gene in the context of the whole organism and the phenomena of gene activation, expression, and interaction. This technology has also been used to produce models for various diseases in humans and other animals and is amongst the most powerful tools available for the study of genetics, and the understanding of genetic mechanisms and function.

From an economic perspective, the use of transgenic technology to convert animals into “protein factories” for the production of specific proteins or other substances of pharmaceutical interest (Gordon et al., 1987, Biotechhnology 5: 1183-1187; Wilmut et al., 1990, Theriogenology 33: 113-123) offers significant advantages over more conventional methods of protein production by gene expression.

Heterologous nucleic acids have been engineered so that an expressed protein may be joined to a protein or peptide that will allow secretion of the transgenic expression product into milk or urine, from which the protein may then be recovered. However, these procedures have had limited success and may require lactating animals, with the attendant costs of maintaining individual animals or herds of large species, including cows, sheep, or goats.

Historically, transgenic animals have been produced almost exclusively by microinjection of the fertilized egg. The pronuclei of fertilized eggs are microinjected in vitro with foreign, i.e., xenogeneic or allogeneic, heterologous DNA or hybrid DNA molecules. The microinjected fertilized eggs are then transferred to the genital tract of a pseudopregnant female (e.g., Krimpenfort et al., in U.S. Pat. No. 5,175,384).

This widely used technique requires equipment to microinject the eggs in vitro and to handle the embryos. Large numbers of fertilized eggs are needed because of the high rate of egg loss due to lysis during microinjection. Moreover, manipulated embryos are less likely to implant and survive in utero. Typically, 300-500 fertilized eggs must be microinjected to produce perhaps three transgenic animals. Consequently, generating large animals with these techniques is prohibitively expensive.

Genetic information also has been transferred to embryos using retroviral vectors (Jaenisch, R., 1976, Proc. Natl. Acad. Sci. USA 73, 1260-1264), but the resulting animals were mosaics with gene insertions at various loci in the genomic nucleic acid of any one animal. The transgenes also were differentially expressed in various tissues of each animal (Jaenisch, R., 1980, Cell 19, 181-188.

Nuclear transfer from cultured cell populations is a further route to genetic modification, whereby donor cells may be sexed, optionally genetically modified, and then selected in culture before their use. The resultant transgenic animal originates from a single transgenic nucleus and mosaics are avoided. The genetic modification is easily transmitted to the offspring. Nuclear transfer from cultured somatic cells also provides a route for directed genetic manipulation of animal species, including the addition or “knock-in” of genes, and the removal or inactivation or “knock-out” of genes or their associated control sequences (Polejaeva et al., 2000, Theriogenology, 53, 117-26). Gene targeting techniques also promise the generation of transgenic animals in which specific genes coding for endogenous proteins have been replaced by exogenous genes such as those coding for human proteins.

Although gene targeting techniques combined with nuclear transfer hold tremendous promise for nutritional and medical applications, current approaches suffer from several limitations, including long generation times between the founder animal and production transgenic herds, and extensive husbandry and veterinary costs. It is therefore desirable to use a system where cultured somatic cells for nuclear transfer are more efficiently employed.

One system that holds potential is the avian reproductive system. The production of an avian egg begins with formation of a large yolk in the ovary of the hen. The unfertilized oocyte or ovum is positioned on top of the yolk sac. After ovulation, the ovum passes into the infundibulum of the oviduct where it is fertilized if sperm are present, and then moves into the oviduct magnum that is lined with tubular gland cells. These cells secrete the egg-white proteins, including ovalbumin, lysozyme, ovomucoid, conalbumin and ovomucin, into the lumen of the magnum where they are deposited onto the avian embryo and yolk.

The hen oviduct offers outstanding potential as a protein bioreactor because of the high levels of protein production, the promise of proper folding and post-translation modification of the target protein, the ease of product recovery, and the shorter developmental period of chickens compared to other potential animal species. As a result, efforts have been made to create transgenic chickens expressing heterologous proteins in the oviduct by means of microinjection of DNA (PCT Publication WO 97/47739). Bosselman et al. in U.S. Pat. No. 5,162,215 describe a method for introducing a replication-defective retroviral vector into a pluripotent stem cell of an unincubated chick embryo, and further describe chimeric chickens whose cells express a heterologous vector nucleic acid sequence. However, the percentage of G₁ transgenic offspring (progeny from vector-positive male G₀ birds) was low and varied between 1% and approximately 8%. DNA injection into avian eggs has so far led to poor and unstable transgene integration (Sang and Perry, 1989, Mol. Reprod. Dev., 1: 98-106) and Naito et al., 1994, Mol. Reprod. Dev. 37: 167-71). In addition, the use of viral vectors imposes limitations upon the success of transgenesis, including limited transgene size and potential viral infection of the offspring. The production of transgenic chickens by DNA microinjection can also be both inefficient and time-consuming.

Another method for generating transgenics is the stable transfection of male germ cells in vitro and their transfer to a recipient testis. PCT Publication WO 87/05325 discloses a method of transferring organic and/or inorganic material into sperm or egg cells by using liposomes. Bachiller et al., (1991, Mol. Reprod. Develop. 30: 194-200) used Lipofectin-based liposomes to transfer DNA into mice sperm, and provided evidence that the liposome transfected DNA was overwhelmingly contained within the sperm's nucleus, although no transgenic mice could be produced by this technique. Nakanishi and Iritani (1993, Mol. Reprod. Develop. 36: 258-261) used Lipofectin-based liposomes to associate heterologous DNA with chicken sperm, which were in turn used to artificially inseminate hens. Although the heterologous DNA was detectable in many of the resultant fertilized eggs, there was no evidence of genomic integration of the heterologous DNA either in the DNA-liposome treated sperm or in the resultant chicks.

Primordial germ cells (PGCs) give rise to embryonic germ cells and ultimately the gametes of mature adults. Late development stage PGCs from stage 27 chick embryos have been isolated (Chang et al., 1997, Cell Biol. Int. 21: 495-499). PGCs have also been isolated from stage XIII to XIV embryos by insertion of a micropipette into the dorsal aorta of the embryo and extraction of blood containing PGCs. (Naito et al, 1994, Mol. Reprod. Dev 39: 153-171; Ponce de Leon et al. in U.S. Pat. No. 6,156,569). Earlier stage PGCs appearing at stages VII-IX (Karagene et al., 1996, Dev. Genet. 19: 290-301) are fewer in number than stage XII-XXVII PGCs and harder to extract. Cell surface markers typical of PGCs, like SSEA-1 and EMA-1, appear only at stage X of differentiation. Isolation of these cells has required culturing on feeder cell layers to allow proliferation and enrichment of the population, which is both time consuming and technically demanding. Once available, however, PGCs offer a means of generating transgenic animals having heterologous genes in the gamete cells. These animals may be interbred to create homozygous animals with heterologous expression and protein production in some or all tissues.

Once a transgenic animal line has been created, the protein expressed from the integrated transgene can be produced in quantity and bears post-translational modifications such as glycosylation that may be necessary for functionality. The exogenous protein can be produced in the white of an avian egg, from which it may be readily purified. The economic advantage of breeding flocks of transgenic birds laying eggs expressing exogenous proteins is significant when compared to more traditional animals, such as cows and goats, producing heterologous protein in milk.

Efficient expression of heterologous proteins requires suitable gene promoter elements to be operably linked to the nucleic acid encoding the protein. For example, a suitable promoter would be that of the chicken lysozyme gene. This gene is highly expressed in the myeloid lineage of hematopoietic cells, and in the tubular glands of the mature hen oviduct (Hauser et al., 1981, Hematol. and Blood Transfusion 26: 175-178; Schutz et al., 1978, Cold Spring Harbor Symp. Quart. Biol. 42: 617-624) and is therefore a suitable candidate for an efficient promoter for heterologous protein production in transgenic animals. A regulatory region of the lysozyme locus that extends over at least 12 kb of DNA 5′ upstream of the gene transcription start site has been described in U.S. patent application Ser. No. 09/922,549, incorporated herein by reference in its entirety, and comprises three enhancer sequences at about −6.1 kb, −3.9 kb, and −2.7 kb (Grewal et al., 1992, Mol. Cell Biol. 12: 2339-2350; Banifer et al., 1996, J. Mol. Med. 74: 663-671), a hormone responsive element (Hecht et al., 1988, E.M.B.O.J. 7: 2063-2073), a silencer element and a complex proximal promoter.

The lysozyme promoter region of chicken is active when transfected into mouse fibroblast cells or chicken promacrophage cells. The presence of a 5′ MAR element increased positional independency of the level of transcription (Stief et al., 1989, Nature 341-345; Sippel et al., pp. 257-265 in “Transgenic Animals: Generation and Use,” ed. L. M. Houdeline).

Another useful promoter is that from the ovalbumin gene, which encodes a 45 kD protein specifically expressed in the tubular gland cells of the magnum of the oviduct (Beato, 1989, Cell 56:335-344). Ovalbumin is the most abundant egg white protein, comprising over 50 percent of the total protein produced by the tubular gland cells, or about 4 grams of protein per large Grade A egg (Gilbert, 1971, “Egg albumen and its Formation” in Physiology and Biochemistry of the Domestic Fowl, Bell and Freeman, eds., Academic Press, London, New York, pp. 1291-1329). The ovalbumin gene and over 20 kb of each flanking region have been cloned and analyzed (Lai et al., 1978, Proc. Natl. Acad. Sci. USA 75:2205-2209; Gannon et al., 1979, Nature 278:428-424; Roop et al., 1980, Cell 19:63-68; and Royal et al., 1975, Nature 279:125-132).

### SUMMARY OF THE INVENTION

The present invention provides methods for isolating a population of avian stage X primordial germ cells from a mixed population suspension of stage X blastodermal cells obtained from an avian embryo. Stromal cells are sedimented from the suspension by gravity, and the population of cells thereby enriched in stage X primordial germ cells is then isolated from the residual medium supernatant. The avian embryo may be from any bird including, for example, chicken, turkey, quail, pheasant, duck, goose and ratite. In one embodiment of the method of the present invention, the avian embryo is a chicken embryo.

The methods of the present invention give isolated cell populations of stage X primordial germ cells that are substantially free of stromal blastoderm cells, have alkaline phosphatase activity, and are positive for Periodic Acid Schiff staining, unlike the stromal cells.

The present invention also provides methods for generating an avian having a heterologous germ cell therein comprising: isolating a population of cells enriched in stage X primordial germ cells; microinjecting this population of cells into a recipient embryo of an avian egg; and allowing the recipient embryo to hatch as a chick having a heterologous germ cell therein. A further embodiment of the method for generating a transgenic avian further comprises the step of allowing the chick having the heterologous germ cell to develop to an adult bird, that may be a chimera, and which has at least some germ cells that are heterologous germ cells.

The present invention further provides methods for the production of transgenic avians capable of expressing a heterologous polypeptide, comprising isolating a population of cells enriched in stage X primordial germ cells; transfecting the isolated stage X primordial germ cells by delivering a heterologous nucleic acid thereto; and then microinjecting the transfected avian stage X primordial germ cells into a recipient embryo of an avian egg. The recipient embryo is allowed to hatch as a chick and mature as an adult bird having a heterologous transfected germ cell therein. The adult bird having the heterologous transfected germ cells may be interbred with, or its sperm used to artificially inseminate, a second adult bird thereby producing transgenic progeny heterozygous for the heterologous nucleic acid. Breeding the heterozygous transgenic progeny bird with a second heterozygous transgenic progeny bird may generate a transgenic progeny bird that is homozygous for the heterologous nucleic acid.

In one embodiment of the methods of the present invention, a heterologous polypeptide encoded by the heterologous nucleic acid is produced by the first heterozygous transgenic bird, the second heterozygous transgenic bird, the homozygous transgenic bird, or any progeny thereof.

In various embodiments of the methods of the present invention for the production of a transgenic avian capable of producing a heterologous polypeptide, the heterologous polypeptide is in the serum of the transgenic bird. In still another embodiment of the present invention, the heterologous polypeptide is delivered to the white of a developing avian egg produced by the transgenic bird.

In other embodiments of the methods of the present invention, for the production of a transgenic avian capable of producing a heterologous polypeptide, the heterologous nucleic acid comprises an expression cassette having a promoter, a transcription termination sequence, and a polypeptide-encoding sequence. In one embodiment of the present invention, the expression cassette comprises a transcription unit encoding a first heterologous polypeptide, and optionally a second heterologous polypeptide, operably linked to an avian specific transcription promoter, a transcription terminator, and optionally an internal ribosome entry site (IRES). In another embodiment of the present invention, the transgenic avian expresses a first and a second transgene encoding a first and a second heterologous polypeptide. The method further comprises the step of combining the first and second heterologous polypeptides, thereby forming a multimeric protein.

In one embodiment of the methods of the present invention, the heterologous polypeptide is selected from the group consisting of a cytokine, erythropoietin, a hormone, an enzyme, a structural protein, and an immunoglobulin. The cytokine may be selected from the group consisting of interferon and granulocyte-macrophage colony-stimulating factor.

It is contemplated to be within the scope of the present invention for the heterologous nucleic acid delivered to the stage X primordial germ cells to be an expression vector such as, but not limited to, a viral vector, a plasmid vector, a linear nucleic acid vector or a combination thereof.

Transcriptional promoters of an expression vector of the present invention may be a constitutively active promoter, such as the early/intermediate cytomegalovirus promoter, or a tissue-specific promoter, preferably a tissue-specific promoter operable in oviduct cells of an avian species including, but not limited to, the promoters of the genes encoding ovalbumin, lysozyme, ovomucoid, ovotransferrin (conalbumin), and ovomucin. Optionally, the transcriptional promoter of an expression vector may be a regulatable promoter.

The transcriptional terminator of an expression vector may further comprise a region encoding a transcriptional terminator, such as a bovine growth hormone transcriptional terminator.

The present invention provides a transfected avian embryonic stage X primordial germ cell, wherein the stage X primordial germ cell is isolated from an avian embryo according to the method of the present invention, and wherein the stage X primordial germ cell is transfected with a heterologous nucleic acid encoding a heterologous protein desired to be expressed by a transgenic avian.

The present invention further provides a transgenic avian producing a heterologous polypeptide in an avian egg, wherein the transgenic avian is produced by the methods of the present invention for transfecting isolated avian stage X primordial germ cells and delivering the transfected stage X primordial germ cells to a recipient avian embryo for development into a mature avian, wherein the mature avian contains at least one heterologous nucleic acid sequence encoding the polypeptide and wherein the polypeptide is delivered to the white of an avian egg by a female of the avian. In one embodiment of this aspect of the present invention, the transgenic avian contains a transcription unit comprising a heterologous nucleotide sequence encoding a desired polypeptide, a transcription promoter, and a transcriptional terminator operatively linked to the nucleotide sequence encoding the polypeptide.

Additional objects and aspects of the present invention will become more apparent upon review of the detailed description set forth below when taken in conjunction with the accompanying figures, which are briefly described as follows.

### BRIEF DESCRIPTION OF THE FIGURES

FIG. 1 demonstrates Periodic Acid Schiff (PAS) staining of PGC and control cells. FIG. 1A shows the negative uptake of stain when sedimented stage X blastodermal stromal cells are stained with PAS. FIG. 1B shows stage X primordial germ cells (PGCs), isolated from a chicken blastoderm, staining positive for PAS.

FIG. 2 shows stage X primordial germ cells (PGCs), isolated from a chicken blastoderm, staining for alkaline phosphatase.

### DETAILED DESCRIPTION OF THE PREFERRED EMBODIMENTS

Reference now will be made in detail to the presently preferred embodiments of the invention, one or more examples of which are illustrated in the accompanying Figures. Each example is provided by way of explanation of the invention, not as limitation of the invention. In fact, it will be apparent to those skilled in the art that various modifications, combinations, additions, deletions, and variations can be made in the present invention without departing from the scope or spirit of the invention. For instance, features illustrated or described as part of one embodiment can be used in another embodiment to yield a still further embodiment. It is intended that the present invention covers such modifications, combinations, additions, deletions and variations as come within the scope of the appended claims and their equivalents.

For convenience, certain terms employed in the specification, examples, and appended claims are collected here.

Definitions

The term “animal” is used herein to include all vertebrate animals, including humans. It also includes an individual animal in all stages of development, including embryonic and fetal stages.

The term “avian” as used herein refers to any species, subspecies or race of organism of the taxonomic class ava, such as, but not limited to, such organisms as chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, Rhode Island, Ausstralorp, Minorca, Amrox, California Gray, Italian Partridge-colored), as well as strains of turkeys, pheasants, quails, duck, ostriches and other poultry commonly bred in commercial quantities.

The term “nucleic acid” as used herein refers to any natural and synthetic linear and sequential arrays of nucleotides and nucleosides, for example cDNA, genomic DNA, mRNA, tRNA, oligonucleotides, oligonucleosides and derivatives thereof. For ease of discussion, such nucleic acids may be collectively referred to herein as “constructs,” “plasmids,” or “vectors.” Representative examples of the nucleic acids of the present invention include bacterial plasmid vectors including expression, cloning, cosmid and transformation vectors such as, but not limited to, pBR322, animal viral vectors such as, but not limited to, modified adenovirus, influenza virus, polio virus, pox virus, retrovirus, and the like, vectors derived from bacteriophage nucleic acid, and synthetic oligonucleotides like chemically synthesized DNA or RNA. The term “nucleic acid” further includes modified or derivatised nucleotides and nucleosides such as, but not limited to, halogenated nucleotides such as, but not only, 5-bromouracil, and derivatised nucleotides such as biotin-labeled nucleotides.

The terms “polynucleotide”, “oligonucleotide”, and “nucleic acid sequence” are used interchangeably herein and include, but are not limited to, coding sequences (polynucleotide(s) or nucleic acid sequence(s) which are transcribed and translated into polypeptide in vitro or in vivo when placed under the control of appropriate regulatory or control sequences); control sequences (e.g., translational start and stop codons, promoter sequences, ribosome binding sites, polyadenylation signals, transcription factor binding sites, transcription termination sequences, upstream and downstream regulatory domains, enhancers, silencers, and the like); and regulatory sequences (DNA sequences to which a transcription factor(s) binds and alters the activity of a gene's promoter either positively (induction) or negatively (repression)). No limitation as to length or to synthetic origin are suggested by the terms described herein.

As used herein the terms “polypeptide” and “protein” refer to a polymer of amino acids of three or more amino acids in a serial array, linked through peptide bonds. The term “polypeptide” includes proteins, protein fragments, protein analogues, oligopeptides and the like. The term “polypeptides” contemplates polypeptides as defined above that are encoded by nucleic acids, produced through recombinant technology, isolated from an appropriate source such as a bird, or are synthesized. The term “polypeptides” further contemplates polypeptides as defined above that include chemically modified amino acids or amino acids covalently or noncovalently linked to labeling ligands.

The term “isolated nucleic acid” as used herein refers to a nucleic acid with a structure (a) not identical to that of any naturally occurring nucleic acid or (b) not identical to that of any fragment of a naturally occurring genomic nucleic acid spanning more than three separate genes, and includes DNA, RNA, or derivatives or variants thereof. The term covers, for example, (a) a DNA which has the sequence of part of a naturally occurring genomic molecule but is not flanked by at least one of the coding sequences that flank that part of the molecule in the genome of the species in which it naturally occurs; (b) a nucleic acid incorporated into a vector or into the genomic nucleic acid of a prokaryote or eukaryote in a manner such that the resulting molecule is not identical to any vector or naturally occurring genomic DNA; (c) a separate molecule such as a cDNA, a genomic fragment, a fragment produced by polymerase chain reaction (PCR), ligase chain reaction (LCR) or chemical synthesis, or a restriction fragment; (d) a recombinant nucleotide sequence that is part of a hybrid gene, i.e., a gene encoding a fusion protein, and (e) a recombinant nucleotide sequence that is part of a hybrid sequence that is not naturally occurring. Isolated nucleic acid molecules of the present invention can include, for example, natural allelic variants as well as nucleic acid molecules modified by nucleotide deletions, insertions, inversions, or substitutions such that the resulting nucleic acid molecule still essentially encodes a lysozyme gene expression control region or a variant thereof of the present invention. The techniques used to isolate and characterize the nucleic acids and proteins of the present invention are well known to those of skill in the art and standard molecular biology and biochemical manuals may be consulted to select suitable protocols without undue experimentation. See, for example, Sambrook et al. (1989), “Molecular Cloning: A Laboratory Manual,” 2nd ed., Cold Spring Harbor Press, the content of which is herein incorporated by reference in its entirety.

The term “gene” or “genes” as used herein refers to nucleic acid sequences (including both RNA or DNA) that encode genetic information for the synthesis of a whole RNA, a whole protein, or any portion of such whole RNA or whole protein. Genes that are not naturally part of a particular organism's genome are referred to as “foreign genes”, “heterologous genes” or “exogenous genes” and genes that are naturally a part of a particular organism's genome are referred to as “endogenous genes”. The term “gene product” refers to RNAs or proteins that are encoded by the gene. “Foreign gene products” are RNA or proteins encoded by “foreign genes” and “endogenous gene products” are RNA or proteins encoded by endogenous genes. “Heterologous gene products” are RNAs or proteins encoded by “foreign, heterologous or exogenous genes” and which, therefore, are not naturally expressed in the cell.

The terms “transcription regulatory sequences” and “gene expression control regions” as used herein refer to nucleotide sequences that are associated with a gene nucleic acid sequence and which regulate the transcriptional expression of the gene. Exemplary transcription regulatory sequences include enhancer elements, hormone response elements, steroid response elements, negative regulatory elements, and the like. The “transcription regulatory sequences” may be isolated and incorporated into a vector nucleic acid to enable regulated transcription in appropriate cells of portions of the vector DNA. The “transcription regulatory sequence” may precede, but is not limited to, the region of a nucleic acid sequence that is in the region 5′ of the end of a protein coding sequence that may be transcribed into mRNA. Transcriptional regulatory sequences may also be located within a protein coding region, in regions of a gene that are identified as “intron” regions, or may be in regions of nucleic acid sequence that are in the region of nucleic acid.

The term “promoter” as used herein refers to the DNA sequence that determines the site of transcription initiation from an RNA polymerase. A “promoter-proximal element” may be a regulatory sequence within about 200 base pairs of the transcription start site. A “magnum-specific” promoter, as used herein, is a promoter that is primarily or exclusively active in the tubular gland cells of the magnum. Useful promoters also include exogenously inducible promoters. These are promoters that can be “turned on” in response to an exogenously supplied agent or stimulus, which is generally not an endogenous metabolite or cytokine. Examples include an antibiotic-inducible promoter, such as a tetracycline-inducible promoter, a heat-inducible promoter, a light-inducible promoter, or a laser inducible promoter. (e.g., Halloran et al., 2000, Development 127: 1953-1960; Gemer et al., 2000, Int. J. Hyperthermia 16:171-81; Rang and Will, 2000, Nucleic Acids Res. 28: 1120-5; Hagihara et al., 1999, Cell Transplant. 8: 4314; Huang et al., 1999, Mol. Med. 5: 129-37; Forster et al., 1999, Nucleic Acids Res. 27: 708-10; Liu et al., 1998, Biotechniques 24: 624-8, 630-2, the contents of which are incorporated herein by reference in their entireties).

The term “coding region” as used herein refers to a continuous linear arrangement of nucleotides that may be translated into a protein. A full length coding region is translated into a full length protein; that is, a complete protein as would be translated in its natural state absent any post-translational modifications. A full length coding region may also include any leader protein sequence or any other region of the protein that may be excised naturally from the translated protein.

The terms “operably” or “operatively linked” refer to the configuration of the coding and control sequences so as to perform the desired function. Thus, control sequences operably linked to a coding sequence are capable of effecting the expression of the coding sequence. A coding sequence is operably linked to or under the control of transcriptional regulatory regions in a cell when DNA polymerase will bind the promoter sequence and transcribe the coding sequence into mRNA that can be translated into the encoded protein. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from said RNA nucleic acid molecule to give a protein or polypeptide or a portion thereof.

The term “nucleic acid vector” as used herein refers to a natural or synthetic single or double stranded plasmid or viral nucleic acid molecule that can be transfected or transformed into cells and replicate independently of, or within, the host cell genome. A circular double stranded plasmid can be linearized by treatment with an appropriate restriction enzyme based on the nucleotide sequence of the plasmid vector. A nucleic acid can be inserted into a vector by cutting the vector with restriction enzymes and ligating the pieces together. The nucleic acid molecule can be RNA or DNA.

The term “expression vector” as used herein refers to a nucleic acid vector that comprises the lysozyme gene expression control region operably linked to a nucleotide sequence coding at least one polypeptide. As used herein, the term “regulatory sequences” includes promoters, enhancers, and other elements that may control gene expression. Standard molecular biology textbooks, such as Sambrook et al. eds “Molecular Cloning: A Laboratory Manual,” 2nd ed., Cold Spring Harbor Press (1989), may be consulted to design suitable expression vectors that may further include an origin of replication and selectable gene markers. It should be recognized, however, that the choice of a suitable expression vector and the combination of functional elements therein depends upon multiple factors including the choice of the host cell to be transformed and/or the type of protein to be expressed.

The term “transfecting agent” as used herein refers to a composition of matter added to the genetic material for enhancing the uptake of heterologous DNA segment(s) into a eukaryotic cell, preferably an avian cell, and more preferably a chicken male germ cell. The enhancement is measured relative to the uptake in the absence of the transfecting agent. Examples of transfecting agents include adenovirus-transferrin-polylysine-DNA complexes. These complexes generally augment the uptake of DNA into the cell and reduce its breakdown during its passage through the cytoplasm to the nucleus of the cell. These complexes can be targeted to the male germ cells using specific ligands that are recognized by receptors on the cell surface of the germ cell, such as the c-kit ligand or modifications thereof. Other preferred transfecting agents include but are not limited to lipofectin, lipfectamine, DIMRIE C, Supeffect, and Effectin (Qiagen), unifectin, maxifectin, DOTMA, DOGS (Transfectam; dioctadecylamidoglycylspermine), DOPE (1,2-dioleoyl-sn-glycero-3-phosphoethanolamine), DOTAP (1,2-dioleoyl-3-trimethylammonium propane), DDAB (dimethyl dioctadecytammonium bromide), DHDEAB (N,N-di-n-hexadecyl-N, N-dihydroxyethyl ammonium bromide), HDEAB (N-n-hexadecylN,N-dihydroxyethylammonium bromide), polybrene, or poly(ethylenimine) (PEI). These non-viral agents have the advantage that they can facilitate stable integration of heterologous DNA sequences into the vertebrate genome, without size restrictions commonly associated with virus-derived transfecting agents.

The terms “transformation” and “transfection” as used herein refer to the process of inserting a nucleic acid into a host. Many techniques are well known to those skilled in the art to facilitate transformation or transfection of a nucleic acid into a prokaryotic or eukaryotic organism. These methods involve a variety of techniques, such as treating the cells with high concentrations of salt such as, but not only a calcium or magnesium salt, an electric field, detergent, or liposome mediated transfection, to render the host cell competent for the uptake of the nucleic acid molecules, and by such methods as sperm-mediated and restriction-mediated integration.

The term “recombinant cell” refers to a cell that has a new combination of nucleic acid segments that are not covalently linked to each other in nature. A new combination of nucleic acid segments can be introduced into an organism using a wide array of nucleic acid manipulation techniques available to those skilled in the art. A recombinant cell can be a single eukaryotic cell, or a single prokaryotic cell, or a mammalian cell. The recombinant cell may harbor a vector that is extragenomic. An extragenomic nucleic acid vector does not insert into the cell's genome. A recombinant cell may further harbor a vector or a portion thereof that is intragenomic. The term intragenomic defines a nucleic acid construct incorporated within the recombinant cell's genome.

The terms “recombinant nucleic acid” and “recombinant DNA” as used herein refer to combinations of at least two nucleic acid sequences that are not naturally found in a eukaryotic or prokaryotic cell. The nucleic acid sequences may include, but are not limited to, nucleic acid vectors, gene expression regulatory elements, origins of replication, suitable gene sequences that when expressed confer antibiotic resistance, protein-encoding sequences and the like. The term “recombinant polypeptide” is meant to include a polypeptide produced by recombinant DNA techniques such that it is distinct from a naturally occurring polypeptide either in its location, purity or structure. Generally, such a recombinant polypeptide will be present in a cell in an amount different from that normally observed in nature.

As used herein, the term “transgene” means a nucleic acid sequence (encoding, for example, a human interferon polypeptide) that is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene according to the present invention will include one or more transcriptional regulatory sequences, polyadenylation signal sequences and any other nucleic acid, such as introns, that may be necessary for optimal expression of a selected nucleic acid.

As used herein, a “transgenic animal” is any avian species, including the chicken, in which one or more of the cells of the avian may contain heterologous nucleic acid introduced by way of human intervention, such as by transgenic techniques well known in the art. The nucleic acid is introduced into a cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animal, the transgene causes cells to express a recombinant form of the subject polypeptide, e.g. either agonistic or antagonistic forms, or in which the gene has been disrupted. The terms “chimeric animal” or “mosaic animal” are used herein to refer to animals in which the recombinant gene is found, or in which the recombinant is expressed in some but not all cells of the animal. The term “tissue-specific chimeric animal” indicates that the recombinant gene is present and/or expressed in some tissues but not others.

The term “male germ cells” as used herein refers to spermatozoa (i.e., male gametes) and developmental precursors thereof. In fetal development, primordial germ cells are thought to arise from the embryonic ectoderm, and are first seen in the epithelium of the endodermal yolk sac at the E8 stage. From there they migrate through the hindgut endoderm to the genital ridges. In the sexually mature male vertebrate animal, there are several types of cells that are precursors of spermatozoa, and which can be genetically modified, including the primitive spermatogonial stem cells, known as AO/As, which differentiate into type B spermatogonia. The latter further differentiate to form primary spermatocytes, and enter a prolonged meiotic prophase during which homologous chromosomes pair and recombine. Useful precursor cells at several morphological/developmental stages are also distinguishable: preleptotene spermatocytes, leptotene spermatocytes, zygotene spermatocytes, pachytene spermatocytes, secondary, spermatocytes, and the haploid spermatids. The latter undergo further morphological changes during spermatogenesis, including the reshaping of their nucleus, the formation of aerosome, and assembly of the tail. The final changes in the spermatozoon (i.e., male gamete) take place in the genital tract of the female, prior to fertilization.

The term “primordial germ cells” as used herein refers to embryonic stem cells that develop into the germ cells of the late stage embryo and hence into the gamete-producing cells of the sexually mature adult animal. Primordial germ cells of an avian appear at stages VII-IX of embryo development. At stage X they begin to develop specific surface markers SSEA-1 and EMA-1. At stages XI-XIV the cells translocate from the ventral surface of the area pellucida to the dorsal hypoblast.

The term “stromal cell” as used herein refers to those cells of an embryonic tissue that are not primordial germ cells or germ cells.

The term “isolated” as used herein refers to a tissue such as a blastoderm that has been removed from its natural environment, such as an avian egg. Alternatively, the term “isolated” as used herein can refer to a population of cells such as stage X primordial germ cells substantially free of other cell types including those stromal cells that naturally coexist with primordial germ cells in a stage X avian embryo.

The term “substantially free” as used herein refers to a population of cells wherein the majority of cells are stage X primordial germ cells.

The term “enriched” as used herein refers to a population of primordial germ cells substantially or completely free of non-primordial germ (stromal) cells.

Following long-standing patent law convention, the terms “a” and “an” as used herein, including the claims, mean “one or more.”

Abbreviations

Abbreviations used in the present specification include the following: aa, amino acid(s); bp, base pair(s); cDNA, DNA complementary to RNA; nt, nucleotide(s); SSC, sodium chloride-sodium citrate; DMSO, dimethyl sulfoxide; MAR; matrix attachment region; TPLSM, two photon laser scanning microscopy; REMI, restriction enzyme mediated integration; DMEM, Dulbecco's Modified Eagles Medium; PGC, primordial germ cell.

The present invention provides methods for isolating avian stage X primordial germ cells that may be genetically modified and then microinjected into the blastoderm of a recipient egg. The recipient embryo is then allowed to develop into a viable chick. The hatchling can be a chimeric bird having at least some germ cells derived from the microinjected stage X primordial germ cells. It is further contemplated that isolated primordial germ cells may be transfected ex vivo so that the chimeric bird may have a population of germ cells that comprise heterologous nucleic acid therein. The heterologous nucleic acid may encode a polypeptide desired to be produced by a transgenic bird.

The methods of the present invention isolate populations of cells enriched for primordial germ cells from stage X avian blastoderms. In these methods, the blastoderm of a stage X embryo comprising stromal and primordial germ cells is isolated by dissection from a freshly laid egg and disrupted to yield viable individual or loosely clumped blastodermal cells that include a subpopulation of primordial germ cells. The dissected blastoderm may be disrupted by any method that does not negatively impact the viability of the cells released from the tissue. Suitable methods include, but are not limited to, proteolytic digestion using, for example, trypsin, chymotrypsin, elastase, collagenase or the like and combinations thereof. The blastoderm may be treated with a chelating agent such as, for example, ethylenediaminetetracetate, sodium salt (EDTA) or the like or a combination of the protease and chelating agent. It is further contemplated, however, that any other tissue disruptive method may be employed, including mechanical methods, that will release individual stromal cells and PGCs into a culture medium or buffer and will not significantly decrease the viability or proliferative capacity of the cells. A single blastoderm can give about 5×10⁴ cells that are suspended in a culture medium such as, for example, DMEM. The cell suspension may be placed in a suitably-sized culture plate well. An optional coverslip may be placed in the bottom of the well to collect sedimented cells for microscopic examination.

Incubation of the cell suspension for about 24 hours allows stromal non-primordial germ cells to sediment to the bottom of the culture plate well. The primordial germ cells (PGCs), which are now substantially free of stromal cells, remain in suspension in the medium, thereby comprising a population enriched in stage X primordial germ cells.

The cell suspension may then be centrifuged or filtered to concentrate the enriched stage X PGC population. A typical yield of stage X PGCs from a single blastoderm is between about 200 and about 300 PGCs. Stage X PGCs can be identified and the degree of isolation from other blastodermal cells determined by a positive Periodic Acid Schiff (PAS) staining reaction, and the presence of alkaline phosphatase activity. Stromal cells are negative for both tests.

Stage X primordial germ cells isolated by the methods of the present invention are suitable target cells for ex vivo transfection to generate transgenic primordial germ cells having a heterologous nucleic acid therein. Transfected avian embryonic primordial germ cells can be reintroduced by, for example, microinjection into or below the blastodermal layer of a viable stage X avian embryo and then incubated to produce a transgenic chicken (or other avian species) that will carry the transgene in the its germ-line tissue.

A variety of vectors useful in carrying out the methods of the present invention are described herein. The introduction of a vector nucleic acid into the isolated stage X PGCs can be performed with embryonic PGCs that are either freshly isolated or in culture. The transgenic cells are then typically injected into the subgerminal cavity beneath a recipient blastoderm in an egg. These vectors may be used for stable introduction of a heterologous coding sequence into the genome of an avian PGC. Suitable vectors may be used that allow expression of exogenous proteins in specific tissues of an avian, and in the oviduct in particular. Vectors can also be selected that allow a hen to produce eggs that contain a heterologous protein, particularly in the white of the egg.

Vectors especially useful for transfecting and generating random, stable integration into the avian genome may contain a coding sequence and a magnum-specific promoter in operational and positional relationship to express the coding sequence in the tubular gland cell of the magnum of the avian oviduct. For instance, the promoter may be derived from the promoter regions of the ovalbumin, lysozyme, conalbumin, ovomucoid, or ovomucin genes. Alternatively, the promoter may be a promoter that is largely, but not entirely, specific to the magnum, such as the lysozyme promoter.

Transfection of the PGCs may be mediated by any number of methods known to those of ordinary skill in the art. The introduction of the vector to the cell may be aided by first mixing the nucleic acid with polylysine or cationic lipids which help facilitate passage across the cell membrane. However, introduction of the vector into a cell can be achieved through the use of a delivery vehicle such as a liposome or a virus. Viruses which may be used to introduce the vectors of the present invention into a stage X PGC include, but are not limited to, retroviruses, adenoviruses, adeno-associated viruses, herpes simplex viruses, and vaccinia viruses. In one method of transfecting stage X PGCs, a packaged retroviral-based vector is used to deliver the vector into recipient cells so that the vector is integrated into the avian genome.

Once transfected with a heterologous nucleic acid, the stage X primordial germ cell population may be delivered to a recipient blastoderm by microinjection and the recipient egg resealed and allowed to develop and hatch a viable chick. Typically, the hatchling will be a chimera wherein at least some germ cells of the chick will have originated from the injected transfected PGCs. Therefore, with transfected PGCs at least some chick germ cells will also have heterologous nucleic acid. It is anticipated that the heterologous nucleic acid may form stable transfectants, whereby the heterologous nucleic acid is incorporated into the genome of the recipient avian cells. The transgenic germ cells of the present invention can, therefore, generate sperm cells comprising a heterologous nucleic acid. Subsequent breeding can produce heterozygous or homozygous birds wherein all cells carry at least one copy of the heterologous nucleic acid. For example, the chimeric hatchling may be bred with a bird not having the heterologous nucleic acid, thereby producing at least one heterozygous offspring.

Cross-breeding heterozygous offspring can result in homozygous and heterozygous birds with respect to the heterologous nucleic acid, according to classic Mendelian inheritance. Progeny, and subsequent generations therefrom, of the initial chimeric bird may have the heterologous nucleic acid in all cells including, but not limited to, germ cells. It is contemplated, however, that if the heterologous nucleic acid, when introduced to the enriched stage X PGCs, does not form integrated stable transfectants, that some progeny birds will be chimeric birds having some cells not having the heterologous nucleic acid therein. It is further contemplated for transgenic birds to be bred by natural coitus or by isolating mature sperm from a male bird and artificially inseminating a recipient female, using techniques well known to those of skill in the art.

The heterologous nucleic acid may encode a heterologous polypeptide desired to be expressed by a mature transgenic bird. For this purpose, the present invention provides heterologous nucleic acids comprising an expression cassette wherein the polypeptide-encoding nucleic acid can be operably linked to a transcription controlling region such as a tissue-specific promoter. Suitable promoters include, but are not limited to, avian oviduct specific promoters, viral promoters, such as the CMV promoter and the like. It is contemplated that the promoter operably linked to the polypeptide encoding nucleic acid will allow for expression of the polypeptide in a heterozygous or homozygous transgenic avian. The desired heterologous protein may be expressed into the serum of the transgenic bird or, for example, in the case of oviduct cells, into the white of a developing egg.

Recombinant Nucleic Acids, and Expression Thereof, Under the Control of Avian Tissue-Specific Promoters

Recombinant expression vectors can be designed for the expression of the encoded proteins in eukaryotic cells. Useful vectors may comprise constitutive or inducible promoters to direct expression of either fusion or non-fusion proteins. With fusion vectors, a number of amino acids are usually added to the expressed target gene sequence such as, but not limited to, a protein sequence for thioredoxin. A proteolytic cleavage site may further be introduced at a site between the target recombinant protein and the fusion sequence. Additionally, a region of amino acids such as a polymeric histidine region may be introduced to allow binding of the fusion protein to metallic ions, such as nickel, bonded to a solid support, and thereby allow purification of the fusion protein. Once the fusion protein has been purified, the cleavage site allows the target recombinant protein to be separated from the fusion sequence. Enzymes suitable for use in cleaving the proteolytic cleavage site include, but are not limited to, Factor Xa and thrombin. Fusion expression vectors that may be useful in the present invention include pGex (Amrad Corp., Melbourne, Australia), pRIT5 (Pharmacia, Piscataway, N.J.) and pMAL (New England Biolabs, Beverly, Mass.), that fuse glutathione S-transferase, protein A, or maltose E binding protein, respectively, to the target recombinant protein.

Expression of a foreign gene can be obtained using eukaryotic host cells such as avian cells. The use of eukaryotic host cells permit partial or complete post-translational modification such as, but not only, glycosylation and/or the formation of the relevant inter- or intra-chain disulfide bonds. Examples of vectors useful for expression in the chicken Gallus gallus include pYepSecl as in Baldari et al., 1987, E.M.B.O.J., 6: 229-234 and pYES2 (Invitrogen Corp., San Diego, Calif.), incorporated herein by reference in their entireties.

The recombinant DNA nucleic acid molecules of the present invention can be delivered to cells using conventional recombinant DNA technology. The recombinant DNA molecules may be inserted into a cell to which the recombinant DNA molecule is heterologous (i.e. not normally present). Alternatively, as described more fully below, the recombinant DNA molecule may be introduced into cells which normally contain the recombinant DNA molecule, for example, to correct a deficiency in the expression of a polypeptide, or where over-expression of the polypeptide is desired.

For expression in heterologous systems, the heterologous DNA molecule is inserted into an expression system or vector of the present invention in proper sense orientation and correct reading frame. The vector may contain the necessary elements for the transcription and translation of the inserted protein-coding sequences such as, for example, an isolated lysozyme gene expression control region, an ovalbumin promoter, an artificial promoter construct and the like.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced to a cell by means of transformation and replicated in cultures, including eukaryotic cells grown in tissue culture.

One aspect of the present invention, therefore, is an expression vector suitable for delivery to a recipient cell for expression of the vector therein. It is contemplated to be within the scope of the present invention for the expression vector to comprise any suitable avian tissue-specific or tissue-restricted promoter, such as an avian lysozyme gene expression control region, an ovalbumin promoter, an ovomucoid promoter, an artificial promoter construct and the like, operably linked to a nucleic acid insert encoding a polypeptide, and optionally a polyadenylation signal sequence. The expression vector of the present invention may further comprise a bacterial plasmid sequence, a viral nucleic acid sequence, or fragments or variants thereof that may allow for replication of the vector in a suitable host.

Nucleic acid sequences or derivative or truncated variants thereof, may be introduced into viruses, such as a vaccinia virus. Methods for making a viral recombinant vector useful for expressing a protein under the control of the lysozyme promoter are analogous to the methods disclosed in U.S. Pat. Nos. 4,603,112; 4,769,330; 5,174,993; 5,505,941; 5,338,683; 5,494,807; 4,722,848; Paoletti, E. Proc. Natl. Acad. Sci. 93: 11349-11353 (1996); Moss, B., Proc. Natl. Acad. Sci. 93: 11341-11348 (1996); Roizman, Proc. Natl. Acad. Sci. 93: 11307-11302 (1996); Frolov et al., Proc. Natl. Acad. Sci. 93: 11371-11377 (1996); Grunhaus et al. Seminars in Virology 3: 237-252 (1993) and U.S. Pat. Nos. 5,591,639; 5,589,466; and 5,580,859 relating to DNA expression vectors, inter alia, the contents of which are incorporated herein by reference in their entireties.

Recombinant viruses can also be generated by transfection of plasmids into cells infected with virus. Suitable vectors include, but are not limited to, viral vectors such as lambda vector system λgt11, λgt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript 11 SK+/− or KS+/− (see “Stratagene Cloning Systems” Catalog (1993) from Stratagene, La Jolla, Calif., hereby incorporated by reference in its entirety), pQE, plH821, pGEX, pET series (see Studier, F. W. et al. (1990) “Use of T7 RNA Polymerase to Direct Expression of Cloned Genes” Gene Expression Technology, vol. 185, which is hereby incorporated by reference) and any derivatives thereof. Recombinant molecules can be introduced into cells via transformation, particularly transduction, conjugation, mobilization, or electroporation. The DNA sequences are cloned into the vector using standard cloning procedures in the art, as described by Sambrook et al., Molecular Cloning: A Laboratory Manual. 2nd ed., Cold Spring Harbor Laboratory, Cold Spring Harbor, N.Y. (1989), hereby incorporated by reference in its entirety.

A variety of host-vector systems may be utilized to express the protein-encoding sequence(s). Primarily, the vector system must be compatible with the host cell used. The use of eukaryotic recipient host cells permits partial or complete post-translational modification such as, but not only, glycosylation and/or the formation of the relevant inter- or intra-chain disulfide bonds. Host-vector systems include, but are not limited to, the following: bacteria transformed with bacteriophage DNA, plasmid DNA, or cosmid DNA; microorganisms such as yeast containing yeast vectors; vertebrate cell systems infected with virus (e.g., vaccinia virus, adenovirus, etc.); insect cell systems infected with virus (e.g., baculovirus) or avian embryonic cells inoculated with the recombinant nucleic acid. The expression elements of these vectors vary in their strength and specificities. Depending upon the host-vector system utilized, any one of a number of suitable transcription and translation elements can be used.

Once an avian tissue-specific promoter and a nucleic acid encoding a heterologous protein of the present invention have been cloned into a vector system, it is ready to be incorporated into a host cell. Such incorporation can be carried out by the various forms of transformation noted above, depending upon the vector/host cell system. It is contemplated that the incorporation of the DNA of the present invention into a recipient cell may be by any suitable method such as, but not limited to, liposomal transfer, viral transfer, electroporation, gene gun insertion, microinjection and the like. Suitable host cells include, but are not limited to, bacteria, virus, yeast, mammalian cells, and the like. In particular, the present invention contemplates the use of recipient avian stage X primordial germ cells derived from such birds as the chicken or quail.

It is contemplated that the transfected cell according to the present invention may be transiently transfected, whereby the transfected recombinant DNA or expression vector may not be integrated into the genomic nucleic acid. It is further contemplated that the transfected recombinant DNA or expression vector may be stably integrated into the genomic DNA of the recipient cell, thereby replicating with the cell so that each daughter cell receives a copy of the transfected nucleic acid. It is still further contemplated for the scope of the present invention to include a transgenic animal producing a heterologous protein expressed from a transfected nucleic acid according to the present invention.

In one embodiment of the present invention, the transgenic animal is an avian selected from a turkey, duck, goose, quail, pheasant, ratite, and ornamental bird or a feral bird. In another embodiment, the avian is a chicken and the heterologous polypeptide produced under the transcriptional control of the avian promoter according to the present invention is produced in the white of an egg. In yet another embodiment of the present invention, the heterologous polypeptide is produced in the serum of a bird.

Codon-Optimized Gene Expression

Another aspect of the present invention is to provide nucleic acid sequences encoding heterologous polypeptides that are codon-optimized for expression in avian cells, and derivatives and fragments thereof.

When a recombinant DNA is to be delivered to a recipient cell for expression therein, the sequence of the nucleic acid sequence may be modified so that the codons are optimized for the codon usage of the recipient species. For example, if the recombinant DNA is transfected into a recipient chicken cell, the sequence of the expressed nucleic acid insert is optimized for chicken codon usage. This may be determined from the codon usage of at least one, and preferably more than one, protein expressed in a chicken cell. For example, the codon usage may be determined from the nucleic acid sequences encoding the proteins ovalbumin, lysozyme, ovomucin and ovotransferrin of chicken as described in Example 8 below.

In one exemplary embodiment of the recombinant DNA of the present invention, a nucleic acid insert encodes the human interferon α2b polypeptide optimized for codon-usage by the chicken. The nucleic acid sequence and origin of the avian codon-optimized human α2b is fully disclosed in U.S. patent application Ser. No. 09/173,864 and PCT Application No. 99/19472, incorporated herein by reference in their entireties. Codon optimization of the sequence is useful in elevating the level of translation in avian eggs.

It is contemplated to be within the scope of the present invention for any nucleic acid encoding a polypeptide to be optimized for expression in avian cells. It is further contemplated that the codon usage may be optimized for a particular avian species used as a source of the host cells. In one embodiment of the present invention, the heterologous polypeptide is encoded using codons optimized for a chicken.

Transgenesis of Stage X Primordial Germ Cells

Using the methods of the present invention, transgenes can be introduced into avian stage X primordial germ cells to produce a transgenic chicken or other avian species carrying the transgene in the genetic material of its germ-line tissue. The methods and vectors of the present invention further are useful in generating transgenic avians capable of expressing heterologous genes in the serum of the avian and/or deposited in an avian egg. The cells useful in the present invention are primordial germ cells (PGCs) isolated from an avian embryo at or around stage X of development. The targeted stage X PGCs may be isolated freshly, maintained in culture, or frozen.

A variety of vectors useful in carrying out the methods of the present invention are described herein. These vectors may be used for the stable introduction of an exogenous coding sequence into the genome of a bird. In alternative embodiments, the vectors may be used to produce heterologous proteins in specific tissues of an avian, and in the oviduct in particular. In still further embodiments, the vectors are used in methods to produce an avian egg containing a heterologous protein.

In one embodiment of the invention, vectors used for transfecting stage X PGCs resulting in random, stable integration of a heterologous coding sequence into the avian genome contain a coding sequence and a magnum-specific promoter in operational and positional relationship to express the coding sequence in the tubular gland cell of the magnum of the avian oviduct. The magnum-specific promoter may optionally be a segment of the ovalbumin promoter region which is sufficiently large to direct expression of the coding sequence in the tubular gland cells. Other exemplary promoters include the promoter regions of the ovalbumin, lysozyme, conalbumin, ovomucoid, or ovomucin genes. Alternatively, the promoter may be a promoter that is largely, but not entirely, specific to the magnum, such as the lysozyme promoter. Other suitable promoters may be artificial constructs such as a combination of nucleic acid regions derived from at least two avian gene promoters.

In an alternative embodiment of the invention, transgenes containing constitutive promoters are used, but the transgenes are engineered so that expression of the transgene effectively becomes magnum-specific. Thus, a method for producing a heterologous protein in an avian oviduct provided by the present invention involves generating a transgenic avian that bears two transgenes in its tubular gland cells. One transgene comprises a first coding sequence operably linked to a constitutive promoter. The second transgene comprises a second coding sequence that is operably linked to a magnum-specific promoter, where expression of the first coding sequence is either directly or indirectly dependent upon the cellular presence of the protein expressed by the second coding sequence.

Optionally, site-specific recombination systems, such as the Cre-loxP or FLP-FRT systems, are utilized to implement the magnum-specific activation of an engineered constitutive promoter. In one embodiment, the first transgene contains an FRT-bounded blocking sequence which blocks expression of the first coding sequence in the absence of FTP, and the second coding sequence encodes FTP. In another embodiment, the first transgene contains a loxP-bounded blocking sequence which blocks expression of the first coding sequence in the absence of the Cre enzyme, and the second coding sequence encodes Cre. The loxP-bounded blocking sequence may be positioned in the 5′ untranslated region of the first coding sequence and the loxP-bounded sequence may optionally contain an open reading frame.

For instance, in one embodiment of the invention, magnum-specific expression is conferred on a constitutive transgene, by linking a cytomegalovirus (CMV) promoter to the coding sequence of the protein to be secreted (CDS). The 5′ untranslated region (UTR) of the coding sequence contains a loxP-bounded blocking sequence. The loxP-bounded blocking sequence contains two loxP sites, between which is a start codon (ATG) followed by a stop codon, creating a short, nonsense open reading frame (ORF). Note that the loxP sequence contains two start codons in the same orientation. Therefore, to prevent them from interfering with translation of the coding sequence after loxP excision, the loxP sites must be orientated such that the ATGs are in the opposite strand.

In the absence of Cre enzyme, the cytomegalovirus promoter drives expression of a small open reading frame (ORF). Ribosomes will initiate at the first ATG, the start codon of the ORF, then terminate without being able to reinitiate translation at the start codon of the coding sequence. To be certain that the coding sequence is not translated, the first ATG is out of frame with the coding sequence's ATG. If the Cre enzyme is expressed in cells containing the CMV-cDNA transgene, the Cre enzyme will recombine the loxP sites, excising the intervening ORF. Translation will begin at the start codon of the coding sequence, resulting in synthesis of the desired protein.

To make this system tissue specific, the Cre enzyme is expressed under the control of a tissue-specific promoter, such as the magnum-specific ovalbumin promoter, in the same cell as the CMV-loxP-coding sequence transgene. Although a truncated ovalbumin promoter may be fairly weak, it is still tissue-specific and will express sufficient amounts of the Cre enzyme to induce efficient excision of the interfering ORF. In fact, low levels of recombinase should allow higher expression of the recombinant protein since it does not compete against coding sequence transcripts for translation machinery.

Alternate methods of blocking translation of the coding sequence include inserting a transcription termination signal and/or a splicing signal between the loxP sites. These can be inserted along with the blocking ORF or alone. In another embodiment of the invention, a stop codon can be inserted between the loxP sites in the signal peptide of the coding sequence. Before recombinase is expressed, the peptide terminates before the coding sequence. After recombinase is expressed (under the direction of a tissue specific promoter), the stop codon is excised, allowing translation of the coding sequence. The loxP site and coding sequence are juxtaposed such that they are in frame and the loxP stop codons are out of frame. Since signal peptides are able to accept additional sequence (Brown et al., 1984, Mol. Gen. Genet 197:351-7), insertion of loxP or other recombinase target sequences (i.e. FRT) is unlikely to interfere with secretion of the desired coding sequence. In one expression vector, the loxP site is present in the signal peptide such that the amino acids encoded by loxP are not present in the mature, secreted protein. Before Cre enzyme is expressed, translation terminates at the stop codon, preventing expression of β-lactamase. After recombinase is expressed (only in magnum cells), the loxP sites recombine and excise the first stop codon. Therefore, β-lactamase is expressed selectively only in magnum cells.

In the aforementioned embodiments, the blocking ORF can be any peptide that is not harmful to chickens. The blocking ORF can also be a gene that is useful for production of the ALV-transduction particles and/or transgenic birds. In one embodiment, the blocking ORF is a marker gene.

For instance, the blocking ORF could be the neomycin resistance gene, which is required for production of transduction particles. Once the transgene is integrated into the chicken genome, the neomycin resistance gene is not required and can be excised.

Alternatively, β-lactamase can be used as the blocking ORF, as it is a useful marker for production of transgenic birds. As an example, the blocking ORF is replaced by β-lactamase and the downstream coding sequence now encodes a secreted biopharmaceutical. β-Lactamase will be expressed in blood and other tissues; it will not be expressed in the magnum after magnum-specific expression of Cre and recombination-mediated excision of β-lactamase, allowing expression of the desired protein.

The Cre and loxP transgenes could be inserted into the chicken genome via mediated transgenesis either simultaneously or separately. Any method of transgenesis that results in stable integration into the chicken genome is suitable including, but not limited to, viral integration. Both the ovalbumin promoter-recombinase and CMV-loxP-CDS transgenes could be placed simultaneously into chickens. However, the efficiencies of transgenesis are low and therefore the efficiency of getting both transgenes into the chicken genome simultaneously is low. In an alternative and preferred method, one flock is produced that carries the magnum-specific promoter/recombinase transgene and a second is produced that carries the CMV-loxP-CDS transgene. The flocks would then be crossed to each other. Hens resulting from this outbreeding will express the coding sequence and only in their magnum.

As mentioned above, the vectors produced according to the methods of the invention may optionally be provided with a 3′ UTR containing a polyadenylation site to confer stability to the RNA produced. In a preferred embodiment, the 3′ UTR may be that of the exogenous gene, or selected from the group consisting of the ovalbumin, lysozyme, or SV40 late region. However, the ovalbumin 3′ UTR is not suitable in a PMGI vector that is to be inserted into the endogenous ovalbumin gene because the addition of ovalbumin sequences to the PMGI vector will interfere with proper targeting.

Viral Host Cell Transformation

Another suitable approach for in vivo introduction of nucleic acid and the associated gene expression control regions into a recipient cell is by use of a viral vector containing nucleic acid, e.g. a cDNA, encoding the gene product. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors are generally understood to be the recombinant gene delivery system of choice for the transfer of heterologous genes in vivo. These vectors provide efficient delivery of genes into cells, and the transferred nucleic acids are stably integrated into the chromosomal DNA of the host. Recombinant retrovirus can be constructed wherein the retroviral coding sequences (gag, pol, env) have been replaced by nucleic acid encoding a polypeptide, thereby rendering the retrovirus replication defective. Protocols for producing recombinant retroviruses and for infecting cells in vitro or in vivo with such viruses can be found in Sections 9.10-9.14 of “Current Protocols in Molecular Biology,” Ausubel et al., eds., Greene Publishing Associates (1989) and other standard laboratory manuals. Examples of suitable retroviruses well known to those skilled in the art include, but are not limited to, pLJ, pZIP, pWE and pEM. Examples of suitable packaging virus lines for preparing both ecotropic and amphotropic retroviral systems include, but are not limited to, psiCrip, psiCre, psi2 and psiAm.

Furthermore, it is possible to limit the infection spectrum of retroviruses, and consequently of retroviral-based vectors, by modifying the viral packaging proteins on the surface of the viral particle (see, for example PCT publications WO 93/25234, WO 94/06920, and WO 94/11524). For instance, strategies for the modification of the infection spectrum of retroviral vectors include coupling antibodies specific for cell surface antigens to the viral env protein (Roux et al., 1989, Proc. Natl. Acad. Sci. 86: 9079-9083; Julan et al., 1992, J. Virol. 73: 3251-3255; and Goud et al., 1983, Virology 163: 251-254), or coupling cell surface ligands to the viral env proteins (Neda et al., 1991, J. Biol. Chem. 266: 14143-14146); the contents of which are incorporated herein by reference in their entireties. Coupling can be in the form of the chemical cross-linking with a protein or other variety (e.g. lactose to convert the env protein to a sialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector. Moreover, use of retroviral gene delivery can be further enhanced by the use of tissue- or cell-specific transcriptional regulatory sequences that control expression of the nucleic acid encoding an immunoglobulin polypeptide of the retroviral vector.

One retrovirus useful for randomly introducing a transgene into the avian genome is the replication-deficient ALV retrovirus. To produce an appropriate ALV retroviral vector, a pNLB vector is modified by inserting a region of the ovalbumin promoter and one or more exogenous genes between the 5′ and 3′ long terminal repeats (LTRs) of the retrovirus genome. Any coding sequence placed downstream of the ovalbumin promoter will be expressed at high levels and only in the tubular gland cells of the oviduct magnum because the ovalbumin promoter drives the high level of expression of the ovalbumin protein and is only active in the oviduct tubular gland cells. While a 7.4 kb ovalbumin promoter has been found to produce the most active construct when assayed in cultured oviduct tubular gland cells, the ovalbumin promoter must be shortened for use in the retroviral vector. In a preferred embodiment, the retroviral vector comprises a 1.4 kb segment of the ovalbumin promoter; a 0.88 kb segment would also suffice.

Any of the vectors of the present invention may also optionally include a coding sequence encoding a signal peptide that will direct secretion of the protein expressed by the vector's coding sequence from the tubular gland cells of the oviduct. This aspect of the invention effectively broadens the spectrum of exogenous proteins that may be deposited in avian eggs using the methods of the invention. Where an exogenous protein would not otherwise be secreted, the vector bearing the coding sequence is modified to comprise a DNA sequence comprising about 60 bp encoding a signal peptide from the lysozyme gene. The DNA sequence encoding the signal peptide is inserted in the vector such that it is located at the N-terminus of the protein encoded by the cDNA.

Another viral gene delivery system useful in the present invention utilizes adenovirus-derived vectors. The genome of an adenovirus can be manipulated such that it encodes a gene product of interest, but is inactivated in terms of its ability to replicate in a normal lytic viral life cycle (see, for example, Berkner et al., 1988, BioTechniques 6: 616; Rosenfeld et al., 1991, Science 252: 431-434; and Rosenfeld et al., 1992, Cell 68: 143-155; the contents of which are incorporated herein by reference in their entireties. Suitable adenoviral vectors derived from the adenovirus strain Ad type 5 dl324 or other strains of adenovirus (e.g., Ad2, Ad3, Ad7 and the like) are well known to those skilled in the art. The virus particle is relatively stable and amenable to purification and concentration and, as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., retroviral DNA). Most replication-defective adenoviral vectors currently in use, and therefore favored by the present invention, are deleted with respect to parts or all of the viral E1 and E3 genes, but retain as much as 80% of the adenoviral genetic material (see, for example, Jones et al., 1979, Cell 16: 683; Berkner et al., supra; and Graham et al., pp. 109-127 in “Methods in Molecular Biology,” vol. 7, E. J. Murray, ed., Humana Publishing, Clifton, N.J., 1991) (the contents of which are incorporated herein by reference in their entireties). Expression of an inserted nucleic acid encoding a polypeptide including, but not limited to, human interferon α2b, an immunoglobulin, EPO, and GM-CSF can be under the control of, for example, the lysozyme promoter, the ovalbumin promoter, artificial promoter construct sequences and the like.

Yet another viral vector system useful for delivery of the subject nucleic acid encoding, for example, an immunoglobulin polypeptide, is the adeno-associated virus (MV). Vectors containing as little as 300 base pairs of AAV can be packaged and can integrate into a host genome, however, space for heterologous DNA is limited to about 4.5 kb. An AAV vector such as that described by Tratschin et al. (1985, Mol. Cell. Biol. 5: 3251-3260) can be used to introduce DNA into cells. A variety of nucleic acids have been introduced into different cell types using AAV vectors (see, for example, Hermonat et al., 1984, Proc. Natl. Acad. Sci. 81: 6466-6470; Tratschin et al., 1985, Mol. Cell. Biol. 4: 2072-2081; Wondisford et al., 1988, Mol. Endocrinol. 2: 32-39; Tratschin et al., 1984, J. Virol. 51: 611-619; and Flotte et al., 1993, J. Biol. Chem. 268: 3781-3790; the contents of which are incorporated herein incorporated herein by reference in their entireties.

Other viral vector systems that may have application in the methods according to the present invention have been derived from, but are not limited to, herpes virus, vaccinia virus, avian leucosis virus and several RNA viruses.

Non-Viral Expression Vectors

Most non-viral methods of gene transfer rely on normal mechanisms used by eukaryotic cells for the uptake and intracellular transport of macromolecules. In preferred embodiments, non-viral gene delivery systems of the present invention rely on endocytic pathways for the uptake of the subject nucleic acid encoding a polypeptide by the targeted cell. Exemplary gene delivery systems of this type include liposomal derived systems, poly-lysine conjugates, and artificial viral envelopes.

In a representative embodiment of the present invention, a nucleic acid encoding a polypeptide can be entrapped in liposomes bearing positive charges on their surface (e.g., lipofectins) and (optionally) which are tagged with antibodies against cell surface antigens of the target tissue (see, inter alia, PCT publication WO 91/06309; Japanese patent application 1047381; and European patent publication EP-A-43075), incorporated herein by reference in their entireties.

In similar fashion, the gene delivery system comprises an antibody or cell surface ligand that is cross-linked with a gene binding agent such as polylysine (see, for example, PCT publications WO 93/04701, WO 92/22635, WO 92/20316, WO 92/19749, and WO 92/06180)(the contents of which are incorporated herein by reference in their entireties). It will also be appreciated that effective delivery of the subject nucleic acid constructs via receptor-mediated endocytosis can be improved using agents which enhance escape of gene from the endosomal structures. For instance, whole adenovirus or fusogenic peptides of the influenza HA gene product can be used as part of the delivery system to induce efficient disruption of DNA-containing endosomes (Mulligan et al., 1993, Science 260-926; Wagner et al., 1992, Proc. Natl. Acad. Sci. 89: 7934; and Christiano et al., 1993, Proc. Natl. Acad. Sci. 90: 2122; incorporated herein by reference in their entireties). It is further contemplated that a recombinant DNA molecule may be delivered to a recipient host cell by other non-viral methods including by gene gun, microinjection, or the like.

Production of Exogenous Protein

Methods of the invention that can provide for the production of heterologous protein in, for example, the avian oviduct and the production of eggs which contain heterologous protein involve providing a suitable vector and introducing the vector into stage X PGCs so that the vector is integrated into the avian genome. A subsequent step involves deriving a mature transgenic avian from the transgenic PGCs produced in the previous steps. Deriving a mature transgenic avian from the PGCs optionally involves transferring the transgenic PGCs to an embryo and allowing that embryo to develop fully, so that the cells become incorporated into the bird as the embryo is allowed to develop.

A transgenic bird so produced from the transgenic PGCs is known as a germ-line founder. A germ-line founder is a founder that carries the transgene in genetic material of its germ-line tissue, and may also carry the transgene in oviduct magnum tubular gland cells that express the exogenous protein. Therefore, in accordance with the invention, the transgenic bird progeny will have tubular gland cells expressing the exogenous protein and the offspring of the transgenic bird will also have oviduct magnum tubular gland cells that express the exogenous protein. Alternatively, the offspring express a phenotype determined by expression of the exogenous gene in a specific tissue of the avian.

The methods and transgenic avians of the present invention can be used to express, in large yields and at low cost, a wide range of desired proteins including those used as human and animal pharmaceuticals, diagnostics, and livestock feed additives. Proteins such as growth hormones, cytokines, structural proteins and enzymes including human growth hormone, interferon, lysozyme, and β-casein are examples of proteins that are desirably expressed in the oviduct and deposited in eggs according to the invention. Other possible proteins to be produced include, but are not limited to, albumin, α-1 antitrypsin, antithrombin III, collagen, factors VIII, IX, X (and the like), fibrinogen, hyaluronic acid, insulin, lactoferrin, protein C, erythropoietin (EPO), granulocyte colony-stimulating factor (G-CSF), granulocyte macrophage colony-stimulating factor (GM-CSF), tissue-type plasminogen activator (tPA), feed additive enzymes, somatotropin, and chymotrypsin. Immunoglobulins and genetically engineered antibodies, including immunotoxins that bind to surface antigens on human tumor cells and destroy them, can also be expressed for use as pharmaceuticals or diagnostics.

Transgenic Birds

Another aspect of the present invention concerns transgenic birds such as, but not only, chickens and quails that contain at least one transgene and that preferably (though optionally) express the subject nucleic acid encoding a polypeptide in one or more cells in the animal, such as the oviduct cells of the chicken. Suitable methods for the generation of transgenic avians having heterologous DNA incorporated therein are described, for example, in U.S. patent application Ser. No. 09/173,864 and incorporated herein by reference in its entirety. In embodiments of the present invention, therefore, the expression of the transgene is restricted to specific subsets of cells, tissues or developmental stages utilizing, for example, cis-acting sequences that control expression in the desired pattern. Toward this end, tissue-specific regulatory sequences, or tissue-specific promoters, and conditional regulatory sequences can be used to control expression of the transgene in certain spatial patterns. Moreover, temporal patterns of expression can be provided by, for example, conditional recombination systems or prokaryotic transcriptional regulatory sequences. The inclusion of a 5′ MAR region in the novel isolated lysozyme gene expression control region of the present invention may allow the heterologous expression unit to escape the chromosomal positional effect (CPE) and therefore be expressed at a more uniform level in transgenic tissues that received the transgene by a route other than through germ line cells.

Conditional transgenes can be provided using prokaryotic promoter sequences that require prokaryotic proteins to be simultaneous expressed to facilitate expression of the transgene. Operators present in prokaryotic cells have been extensively characterized in vivo and in vitro and can be readily manipulated to place them in any position upstream from or within a gene by standard techniques. Such operators comprise promoter regions and regions that specifically bind proteins such as activators and repressors. One example is the operator region of the lexA gene of E. coli to which the LexA polypeptide binds. Other exemplary prokaryotic regulatory sequences and the corresponding trans-activating prokaryotic proteins are disclosed by Brent and Ptashne in U.S. Pat. No. 4,833,080, and incorporated herein by reference in its entirety. Transgenic animals can be created which harbor the subject transgene under transcriptional control of a prokaryotic sequence that is not appreciably activated by eukaryotic proteins. Breeding of this transgenic animal with another animal that is transgenic for the corresponding prokaryotic trans-activator can permit activation of the nucleic acid encoding an immunoglobulin polypeptide. Moreover, expression of the conditional transgenes can be induced by gene therapy-like methods (such as described above) wherein a gene encoding the trans-activating protein, e.g., a recombinase or a prokaryotic protein, is delivered to the tissue and caused to be expressed, such as in a cell-type specific manner.

Additionally, inducible promoters can be employed, such as the tet operator and the metallothionein promoter that can be induced by treatment with tetracycline and zinc ions, respectively (Gossen et al., 1992, Proc. Natl. Acad. Sci. 89: 5547-5551; and Walden et al., 1987, Gene 61: 317-327; incorporated herein by reference in their entireties.

In the case of an avian, a heterologous polypeptide or polypeptides encoded by the transgenic nucleic acid may be secreted into the oviduct lumen of the mature animal and deposited as a constituent component of the egg white into eggs laid by the animal. It is also contemplated to be within the scope of the present invention for the heterologous polypeptides to be produced in the serum of a transgenic avian. A leaky promoter such as the CMV promoter may be operably linked to a transgene, resulting in expression of the transgene in all tissues of the transgenic avian, resulting in production of a heterologous polypeptide in the serum. Transgenic avians produced by the present invention will have the ability to lay eggs that contain one or more desired heterologous protein(s) or variants thereof.

Generation of Transgenic Avian Zygotes by Restriction Enzyme-Mediated Integration (REMI)

The REMI method for stably integrating heterologous DNA into the genomic DNA of a recipient cell is described by Shemesh et al. in PCT Publication No. WO 99/42569, and incorporated herein by reference in its entirety. This REMI method comprises, in part, an adaptation of the REMI technique disclosed by Schiest and Petes (1991, Proc. Nat. Acad. Sci. U.S.A. 88: 7585-7589 (1991) and Kuspa and Loomis (1992, Proc. Nat Acad. Sci. U.S.A., 89: 8803-8807), both incorporated herein by reference in their entireties.

The REMI method is suitable for introducing heterologous DNA into the genome nucleic acid of an embryonic cell, including the isolated stage X PGCs of the present invention, or somatic cell of an avian.

The heterologous nucleic acid to be integrated into, for example, the PGC nuclear DNA is converted to a linear double stranded DNA possessing single-stranded cohesive ends by contacting the heterologous DNA with a type II restriction enzyme that upon scission, generates such ends. The nucleic acid to be cut can be a circular nucleic acid such as in a plasmid or a viral vector or a linear nucleic acid that possesses at least one recognition and cutting site outside of the genes or regulatory regions critical to the desired post-integration function of the nucleic acid, and no recognition and cutting sites within the critical regions.

Alternatively the heterologous DNA to be integrated into the PGC nuclear DNA can be prepared by chemically and/or enzymatically adding cohesive ends to a linear DNA (see, for example, Sambrook et al., 1989, Molecular Cloning: A Laboratory Manual, 2nd ed., Cold Spring Harbor Laboratory, Cold Spring Harbor, N.Y.; incorporated herein by reference in its entirety). The added cohesive ends must be able to hybridize to the cohesive ends characteristic of a nucleic acid cleaved by a type II restriction endonuclease. Alternatively the cohesive ends can be added by combining the methods based on type II restriction enzyme cutting and chemical and/or enzymatic addition.

According to the present invention, a heterologous nucleic acid encoding at least one polypeptide, and the appropriate restriction enzyme can be introduced into PGCs together or sequentially by way of, for example, electroporation, or lipofection. Preferably electroporation may be used, and most preferably lipofection is used. However, the present invention contemplates that any technique capable of transferring heterologous material into PGCs could be used. It is understood that the heterologous nucleic acid may be integrated into the genome of a recipient cell. It is further understood that the heterologous nucleic acid may not be integrated into the genome of the recipient cell. The combination of REMI as described in the present application, plus a relatively benign method of transferring heterologous material into a cell may result in heterologous nucleic acid being stably integrated into genomic DNA of a high fraction of the treated stage X PGCs.

It is contemplated to be within the scope of the present invention for nucleic acids encoding polypeptides to be derived from any suitable species including, for example, a human, a mouse, a rat, a rabbit, a goat, a sheep, a cow, a horse or a bird. Antibodies may be monoclonal antibodies. It is further within the scope of the present invention for polypeptides to be modified, for example, by exchanging regions within the polypeptides from one animal species for equivalent regions from another species. It is further understood that a polypeptide from one animal species may be combined with a polypeptide from another animal species.

One aspect of the present invention, therefore is methods for isolating a population of avian stage X primordial germ cells, comprising the steps of obtaining an avian egg having a stage X blastoderm, isolating the stage X blastoderm from the avian egg, releasing a population of cells from the isolated stage X blastoderm, wherein the population of cells includes stage X primordial germ cells, incubating the released population of cells in a culture medium, whereby stromal cells sediment from the culture medium, and isolating from the culture medium a population of cells enriched in stage X primordial germ cells. In one embodiment of the method of the present invention, the population of cells is released from an isolated stage X blastoderm by proteolytic digestion of the blastoderm.

In the various embodiments of the methods of the present invention, the avian egg may be obtained from, for example, any of the group consisting of chicken, turkey, quail, pheasant, duck, goose and ratite. In one embodiment of the method of the present invention, the avian egg is a chicken egg.

In one embodiment of the methods of the present invention, the stage X primordial germ cells have alkaline phosphatase activity and are positive for Periodic Acid Schiff (PAS) staining.

Another aspect of the present invention is methods for generating a transgenic avian having a heterologous germ cell therein, comprising the steps of obtaining an avian egg having a stage X blastoderm, isolating the stage X blastoderm from the avian egg, releasing a population of cells from the isolated stage X blastoderm, wherein the population of cells includes embryonic stage X primordial germ cells therein, incubating the released population of cells in a culture medium, whereby stromal cells sediment from the culture medium, isolating from the culture medium a population of cells enriched in stage X primordial germ cells, microinjecting the isolated population of enriched primordial germ cells into a recipient embryo of an avian egg, and allowing the recipient embryo to develop and hatch as a chick having a heterologous germ cell therein.

One embodiment of the method for generating an avian having a heterologous germ cell therein further comprises the step of allowing the chick having the heterologous germ cell to develop to an adult bird. The adult bird may be a chimera, wherein at least some of the germ cells thereof are heterologous germ cells.

In the embodiments of the methods of the present invention for generating an avian having a heterologous germ cell therein, the avian may be selected from the group consisting of chicken, turkey, quail, pheasant, duck, goose and ratite. In one embodiment, the avian is a chicken.

Yet another aspect of the present invention are methods for the production of transgenic avians capable of producing a heterologous polypeptide, comprising the steps of obtaining an avian egg having a stage X blastoderm, isolating the stage X blastoderm from the avian egg, releasing a population of cells from the isolated stage X blastoderm, wherein the population of cells includes stage X primordial germ cells therein, incubating the released population of cells in a culture medium, whereby stromal cells sediment from the culture medium, isolating from the culture medium a population of cells enriched in stage X primordial germ cells, generating a transfected avian stage X primordial germ cell by delivering a heterologous nucleic acid to the population of cells enriched in stage X primordial germ cells, wherein the heterologous nucleic acid comprises an expression cassette, microinjecting the transfected avian stage X primordial germ cell into a recipient embryo of an avian egg, allowing the recipient embryo to hatch as a chick and mature as an adult bird having a heterologous transfected germ cell therein. Breeding the adult bird having a heterologous transfected germ cell therein with a second adult bird may produce a first transgenic progeny bird heterozygous for the heterologous nucleic acid. Subsequently, mating the heterozygous first transgenic progeny bird with a heterozygous second transgenic progeny bird can generate a homozygous or heterozygous transgenic progeny bird depending upon the genotype of the second bird. In the embodiments of the methods of the present invention, the avian may be selected from the group consisting of chicken, turkey, quail, pheasant, duck, goose and ratite. In one embodiment, the avian is a chicken.

In one embodiment of the methods of the present invention for the production of transgenic avians capable of producing a heterologous polypeptide, a heterologous polypeptide encoded by the heterologous nucleic acid can be produced by any of the group consisting of the first heterozygous transgenic bird, the second heterozygous transgenic bird and homozygous transgenic bird, or any progeny thereof.

In one embodiment of the methods of the present invention for the production of transgenic avians capable of producing a heterologous polypeptide, a heterologous polypeptide is in the serum of the transgenic bird. In another embodiment of the methods of the present invention for the production of transgenic avians capable of producing a heterologous polypeptide, the heterologous polypeptide is delivered to the white of a developing avian egg produced by the transgenic bird.

In still another embodiment of the present invention the expression cassette comprises a promoter, a transcription termination sequence and a polypeptide-encoding sequence.

In yet another embodiment of the methods of the present invention for the production of transgenic avians capable of producing a heterologous polypeptide, the expression cassette comprises a transcription unit encoding a first heterologous polypeptide, and optionally a second heterologous polypeptide, operably linked to a avian specific transcription promoter, a transcription terminator, and optionally an internal ribosome entry site (IRES).

In another embodiment of the methods of the present invention for the production of transgenic avians capable of producing a heterologous polypeptide, the transgenic avian expresses a first and a second transgene encoding a first and a second heterologous polypeptide, and wherein the method further comprises the step of combining the first and second heterologous polypeptides, thereby forming a multimeric protein.

In one embodiment of the methods of the present invention, the heterologous polypeptide is selected from the group consisting of a cytokine, hormone, enzyme, structural protein, and immunoglobulin.

In other embodiments of the methods of the present invention, the cytokine may be selected from the group consisting of interferon, interleukin, granulocyte colony-stimulating factor; granulocyte-macrophage colony-stimulating factor; stem cell factor, erythropoietin, thrombopoietin and stem cell factor.

In still other embodiments of the methods of the present invention, the cytokine may be selected from the group consisting of interferon, granulocyte-macrophage colony-stimulating factor, and erythropoietin.

In another embodiment of the methods of the present invention, the hormone is selected from the group consisting of insulin, insulin-like growth factor, growth hormone, and human growth hormone.

It is contemplated to be within the scope of the present invention for the heterologous nucleic acid delivered to the stage X primordial germ cells to be an expression vector such as, but not limited to, a viral vector, a plasmid vector, or a linear nucleic acid vector or a combination thereof. The expression vector may be any suitable viral vector, for example, avian leucosis virus, adenoviral vectors, transferrin-polylysine enhanced adenoviral vectors, human immunodeficiency virus vectors, lentiviral vectors, Moloney murine leukemia virus-derived vectors and the like, and virus-derived DNAs that facilitate polynucleotide uptake by and release into the cytoplasm of germs cells.

Transcriptional promoters of an expression cassette or vector of the present invention may be a constitutively active promoter such as the early/intermediate cytomegalovirus promoter, or a tissue-specific promoter, preferably a tissue-specific promoter operable in oviduct cells of an avian species including, but not limited to, the promoters of the genes encoding ovalbumin, lysozyme, ovomucoid, ovotransferrin (conalbumin), and ovomucin. Optionally, the transcriptional promoter of an expression vector may be a regulatable promoter.

The transcriptional terminator of at least one expression vector may further comprises a region encoding a transcriptional terminator, such as a bovine growth hormone transcriptional terminator.

Another aspect of the present invention is a transgenic avian producing a heterologous polypeptide in an avian egg, wherein the transgenic avian is produced by the methods of the present invention for transfecting an isolated avian stage X germ cell and delivering the transfected stage X primordial germ cell to a recipient avian embryo for development into a mature avian, wherein the mature avian comprises at least one heterologous nucleic acid sequence encoding the polypeptide and wherein the polypeptide is delivered to the white of an avian egg.

In one embodiment of this aspect of the present invention, the transgenic avian comprises a transcription unit comprising a heterologous nucleotide sequence encoding at least one polypeptide, a transcription promoter and a transcriptional terminator operatively linked to the nucleotide sequence encoding at least one polypeptide.

Another aspect of the present invention is a transfected avian embryonic stage X primordial germ cell, wherein the stage X primordial germ cell is isolated from an avian egg according to the method of the present invention, and wherein the stage X primordial germ cell is transfected with a heterologous nucleic acid encoding a heterologous protein desired to be expressed by a transgenic avian. The transfected avian primordial germ cell may be delivered to an avian embryo whereupon the transfected germ cells may become incorporated into the cell mass of the embryo. Subsequent development of the egg will allow the transfected germ cells to give rise to a population of germ cells in a mature bird. Breeding of this bird may result in the generation of transgenic progeny birds capable of expressing the heterologous nucleic acid and thereby delivering a desired heterologous polypeptide to a specific tissue or in the serum of the bird.

Yet another aspect of the present invention is a transgenic avian producing a protein in an avian serum, wherein the transgenic avian comprises at least one heterologous nucleic acid sequence encoding the polypeptide, and wherein an antibody is delivered to the serum of the avian.

In one embodiment of this aspect of the present invention, the transgenic avian comprises a transcription unit comprising a heterologous nucleotide sequence encoding at least one polypeptide, a transcription promoter and a transcriptional terminator operatively linked to the nucleotide sequence encoding at least one polypeptide.

The present invention is further illustrated by the following examples, which are provided by way of illustration and should not be construed as limiting. The contents of all references, published patents and patents cited throughout the present application are hereby incorporated by reference in their entireties.

### EXAMPLE 1

### Isolation of Embryonic Stage X Primordial Germ Cells

Cells were collected from the embryonic stage X blastoderms of freshly laid Barred Rock (BRD) birds. Blastoderms were dissected from the freshly laid eggs and the integrity of the dissected tissue was disrupted by tryptic digestion. Cells (about 50-60×10³ cells per blastoderm) that were released from the stage X blastoderms were suspended in 1×DMEM containing 10% FBS and penicillin/streptomycin solution and plated in 6-well plates. A cover slip was placed in each well to collect cells that settled under gravity for subsequent characterization. After 24 hrs in culture, cells in the supernant were collected by centrifugation (500 rpm at room temperature for approximately 10 minutes) and rinsed once with 1×PBS, yielding the desired cell population highly enriched in embryonic stage X primordial germ cells (PGCs). Typically, a single stage X blastoderm yields between about 200 and about 300 primordial germ cells from a population of about 50,000 blastodermal cells. To identify PGCs, and determine the degree of isolation from other blastodermal cells, cell suspensions were characterized by staining with Periodic Acid Schiff (PAS) and alkaline phosphatase reagents. PGCs are positive when stained with PAS and are positive for alkaline phosphatase activity, while stromal cells are negative for both (Ponce de Leon et al., U.S. Pat. No. 6,156,569; Pain et. al., 1996, Development 122: 2339-2348)

Periodic Acid Schiff (PAS) staining. PGCs in a total volume of 100-200 μl were placed on a slide and then heated over a flame to fix and adhere the cells to the slide. The slides were then processed using the Periodic Acid Schiff (PAS) staining system according to manufacturer's protocol (Sigma Diagnostics, Inc). Cells that had settled down on the cover slips during the isolation of the PGCs were processed in a similar manner as negative controls.

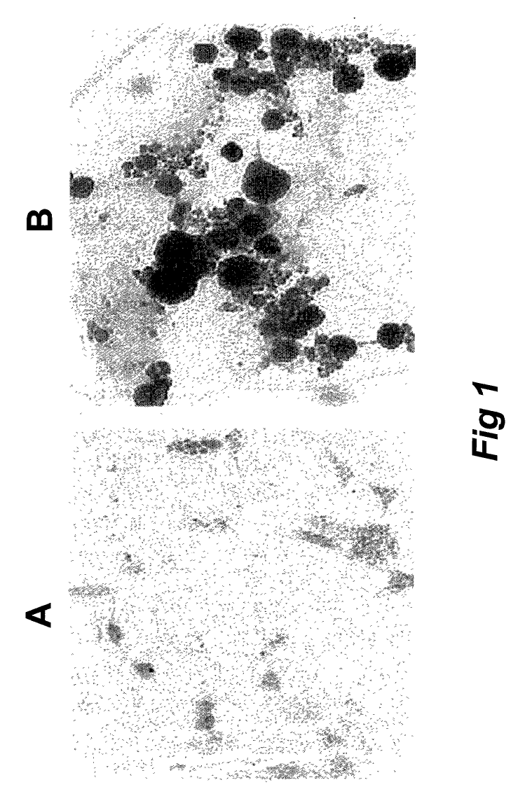

As shown in FIG. 1, cells collected from the enriched supernant stained positive for PAS (FIG. 1A), while control cells were negative for the stain (FIG. 1B).

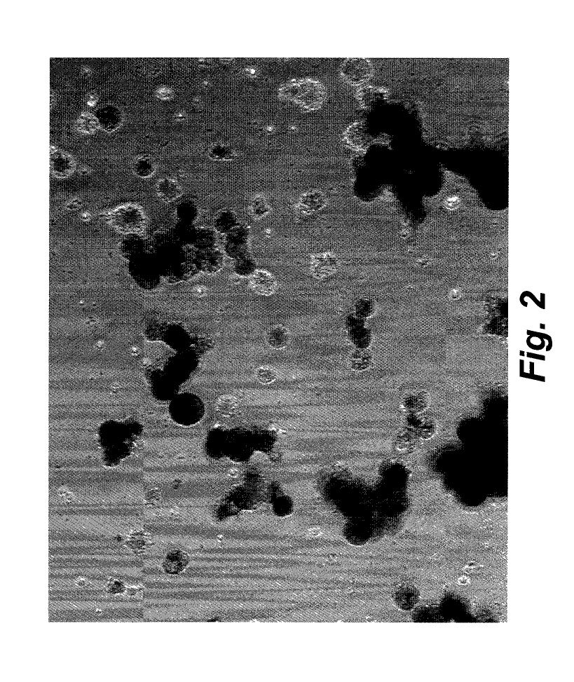

Alkaline Phosphatase (AP) staining. Cells were stained for alkaline phosphatase activity using a Vector Red Alkaline Phosphatase Substrate Kit I (Vector Laboratories) according to the manufacturer's instructions. As shown in FIG. 2, the stage X PGCs in the enriched cell population were positive for alkaline phosphatase activity, in contrast to stromal cells used as negative controls.

The results obtained from staining the enriched PGC fraction, obtained from sedimentation of cells from a blastodermal cell population, demonstrate that the PGC fraction is substantially free of other cell types and subtypes of stromal blastodermal cells.

### EXAMPLE 2

### Germ-Line Testing of the PGCs

To study the potential of the enriched stage X PGCs for germ-line transmission, cells collected from blastoderms of black embryos (Barred Rock) were injected into white embryos, with the transmission of black feather color used to germ-line heritability. In the first experiment, 30 recipient White Leghorn stage X embryos were irradiated to a dosage level of 600 rads and approximately 150 PGC cells, isolated from black Barred Rock (BRD) birds as described in Example 1, were microinjected into each embryo. Eleven chicks hatched with one exhibiting 20% feather color.

In the second experiment, PGC suspensions were collected as described above and passed through a 15 μm filter. Cells were gently washed from the filter and concentrated by centrifugation at 500 rpm at room temperature. Stage X PGCs, isolated from three BRD embryos, were pooled and injected into 29 recipient White Leghorn embryos. Nine chicks hatched, with two of the chicks exhibiting feather chimerism. Both chimeric birds, numbers 2660 and 2661, were males suitable for breeding to BRD hens.

The mature birds displaying chimeric feather color distribution were bred to Barred Rock birds. Because the black phenotype is recessive in chickens, the production of black progeny indicates that the parental chimeras contained germ cells of Barred Rock origin. That is, that at least some of the enriched population of stage X primordial germ cells from black donor blastoderms, injected into white embryonic recipients, developed as germ cells and were passed to progeny. The results are summarized as follows:

TABLE 1
Results of Breeding Chimeric and Barred Rock Birds.

Progeny Feather Color

| | Black | White | Total |
|---|---|---|---|
| Male #2660 | 3 | 17 | 20 |
| Male #2661 | 7 | 12 | 19 |
| Female #2659 | 1 | 8 | 9 |
| Female #2358 | 0 | 10 | 10 |
| Female #2360 | 0 | 11 | 11 |
| Female #2654 | 0 | 7 | 7 |

### EXAMPLE 3

### Production of Fully Transgenic G₁ Chickens

Males are selected for breeding because a single male can give rise to 20 to 30 G₁ offspring per week, as opposed to 6 G₁ offspring per female per week, thereby speeding the expansion of a G₁ transgenic flock. The feed of G₀ males is supplemented with sulfamethazine to accelerate the sexual maturation of male birds, such that they start producing sperm at 10-12 weeks of age, instead of the usual 20-22 weeks. The use of sulfamethazine to decrease time to maturation does not adversely affect the male birds' health or fertility.

Sperm DNA of all males are screened for the presence of the transgene. Sperm are collected and the DNA extracted using Chelex-100. Briefly, 3 μl of sperm and 200 μl of 5% Chelex-100 are mixed, followed by the addition of 2 μl of 10 mg/ml proteinase K and 7 μl of 2 M DTT. Samples are incubated at 56° C. for 30-60 minutes. Samples are boiled for 8 minutes and vortexed vigorously for 10 seconds. After centrifugation at 10 to 15 G for 2-3 minutes, the supernatant is ready for analysis using a PCR technique, Taqman™ analysis, or the like. Using a Taqman™ assay, for example, the extracted DNA is analyzed using a Taqman™ probe and primers complementary to the transgene. It is estimated that 5%, or 4 to 5 chicks, will have the transgene in their sperm DNA.

G₁ offspring will be obtained through breeding germline transgenic males to non-transgenic, White Leghorn females. Hatched chicks are vent-sexed and screened for the presence of the transgene in their blood DNA using a Taqman™ assay, PCR assay, western blot, or the like. Twenty male and female G₁ transgenics will be obtained, in approximately 3-4 weeks. Males will be kept for further breeding and females tested for the presence of the transgene, and expression of the protein coded by that transgene, in the egg white.

What is claimed is:

1. A method for isolating a population of avian stage X primordial germ cells, comprising the steps of:
(a) obtaining an avian egg having a stage X blastoderm;
(b) isolating the stage X blastoderm from the avian egg;
(c) releasing a population of cells from the isolated stage X blastoderm, wherein the population of cells includes stage X primordial germ cells;
(d) incubating the released population of cells in a culture medium, whereby stromal cells sediment from the culture medium; and
(e) isolating from the culture medium a population of cells enriched in stage X primordial germ cells.

2. The method of claim 1, wherein the avian egg is obtained from any of the group consisting of chicken, turkey, quail, pheasant, duck, goose and ratite.

3. The method of claim 1, wherein the avian egg is a chicken egg.

4. The method of claim 1, wherein the stage X primordial germ cells have alkaline phosphatase activity.

5. The method of claim 1, wherein the stage X primordial germ cells are positive for Periodic Acid Schiff staining.

6. The method of claim 1, wherein the population of cells is released from the isolated stage X blastoderm by proteolytic digestion of the blastoderm.

7. A method for generating an avian having a heterologous germ cell therein, comprising the steps of:
(a) obtaining an avian egg having a stage X blastoderm;
(b) isolating the stage X blastoderm from the avian egg;
(c) releasing a population of cells from the isolated stage X blastoderm, wherein the population of cells includes embryonic stage X primordial germ cells therein;
(d) incubating the released population of cells in a culture medium, whereby stromal cells sediment from the culture medium;
(e) isolating from the culture medium a population of cells enriched in stage X primordial germ cells;
(f) delivering the population of cells isolated in step (e) into a recipient avian embryo; and
(g) allowing the recipient embryo to hatch as a chick having a heterologous germ cell therein.

8. The method of claim 7, further comprising the step:
allowing the chick having the heterologous germ cell to develop to an adult bird.

9. The method of claim 7, wherein the chick is a chimera, and wherein at least some of the cells thereof are heterologous germ cells.

10. The method of claim 7, wherein the avian is selected from the group consisting of chicken, turkey, quail, pheasant, duck, goose, and ratite.

11. The method of claim 7, wherein the avian is a chicken.

12. A method for the production of a transfected avian stage X primordial germ cell, comprising the steps of:
(a) obtaining an avian egg having a stage X blastoderm;
(b) isolating the stage X blastoderm from the avian egg;
(c) releasing a population of cells from the isolated stage X blastoderm, wherein the population of cells includes stage X primordial germ cells therein;
(d) incubating the released population of cells in a culture medium, whereby stromal cells sediment from the culture medium;
(e) isolating from the culture medium a population of cells enriched in stage X primordial germ cells; and
(f) transfecting an avian stage X primordial germ cell by delivering a heterologous nucleic acid to the population of cells enriched in stage X primordial germ cells.

13. The method of claim 12, wherein the heterologous nucleic acid comprises an expression cassette.

14. The method of claim 12, wherein the heterologous nucleic acid comprises a vector.

15. The method of claim 13, wherein the expression cassette comprises a promoter, a transcription termination sequence and a polypeptide-encoding sequence.

16. The method of claim 13, wherein the expression cassette comprises a transcription unit encoding a first heterologous polypeptide, and optionally a second heterologous polypeptide, operably linked to a avian specific transcription promoter, a transcription terminator, and optionally an internal ribosome entry site (IRES).

17. The method of claim 14, wherein the vector is selected from the group consisting of a viral vector, a plasmid vector and a linear nucleic acid.

18. The method of claim 12, wherein the avian is selected from the group consisting of chicken, turkey, quail, pheasant, duck, goose and ratite.

19. The method of claim 12, wherein the avian is a chicken.

20. A method for the production of a transgenic avian capable of producing a heterologous protein, comprising the steps of:
(a) obtaining an avian egg having a stage X blastoderm;
(b) isolating the stage X blastoderm from the avian egg;
(c) releasing a population of cells from the isolated stage X blastoderm, wherein the population of cells includes stage X primordial germ cells therein;
(d) incubating the released population of cells in a culture medium, whereby stromal cells sediment from the culture medium;
(e) isolating from the culture medium a population of cells enriched in stage X primordial germ cells;
(f) transfecting avian stage X primordial germ cell by delivering a heterologous nucleic acid to the population of cells enriched in stage X primordial germ cells, wherein the heterologous nucleic acid encodes a polypeptide desired to be expressed by a transgenic avian;
(g) delivering the transfected avian stage X primordial germ cell into a recipient embryo of an avian egg;
(h) allowing the recipient embryo to hatch as a chick and mature as an adult bird having a heterologous transfected germ cell therein; and
(i) breeding the adult bird having a heterologous transfected germ cell therein, thereby producing a transgenic progeny bird having the heterologous nucleic acid therein.

21. The method of claim 20, further comprising the step of breeding the transgenic progeny bird, thereby generating a transgenic progeny bird homozygous for the heterologous nucleic acid.

22. The method of claim 20, further comprising the step of breeding the transgenic progeny bird, thereby generating a transgenic progeny bird heterozygous for the heterologous nucleic acid.

23. The method of claim 20, further comprising the step of expressing the heterologous polypeptide encoded by the heterologous nucleic acid.

24. The method of claim 23, wherein the heterologous polypeptide is expressed in the serum of a transgenic bird.

25. The method of claim 23, wherein the expressed heterologous polypeptide is delivered to the white of a developing avian egg produced by a transgenic bird.

26. The method of claim 20, wherein the heterologous nucleic acid comprises an expression cassette having a promoter, a transcription termination sequence and a polypeptide-encoding sequence.

27. The method of claim 26, wherein the expression cassette has a transcription unit encoding a first heterologous polypeptide, and optionally a second heterologous polypeptide, operably linked to a avian specific transcription promoter, a transcription terminator, and optionally an internal ribosome entry site (IRES).

28. The method of claim 23, wherein the transgenic avian expresses a first and a second transgene encoding a first and a second heterologous polypeptide, and wherein the method further comprises the step of combining the first and second heterologous polypeptides, thereby forming a multimeric protein.

29. The method of claim 20, wherein the avian is selected from the group consisting of chicken, turkey, quail, pheasant, duck, goose and ratite.

30. The method of claim 20, wherein the avian is a chicken.

31. The method of claim 20, wherein the polypeptide is selected from the group consisting of a cytokine, hormone, enzyme, structural protein, and immunoglobulin.

32. The method of claim 31, wherein the cytokine is selected from the group consisting of interferon, interleukin, granulocyte colony-stimulating factor; granulocyte-macrophage colony-stimulating factor; stem cell factor, erythropoietin, thrombopoietin and stem cell factor.

33. The method of claim 31, wherein the cytokine is selected from the group consisting of interferon, granulocyte-macrophage colony-stimulating factor and erythropoietin.

34. The method of claim 31, wherein the hormone is selected from the group consisting of insulin, insulin-like growth factor, growth hormone, and human growth hormone.

35. A transfected avian embryonic stage X primordial germ cell, wherein the stage X primordial germ cell is isolated from an avian egg according to the method of claim 1, and wherein the stage X primordial germ cell is transfected with a heterologous nucleic acid encoding a heterologous protein desired to be expressed by a transgenic avian.

36. A transgenic avian producing a heterologous polypeptide in an avian egg, wherein the transgenic avian is produced by transfecting an isolated avian stage X germ cell, delivering the transfected stage X primordial germ cell to a recipient avian embryo for development into a mature avian having a transfected heterologous germ cell therein, and breeding the mature avian with a second avian to generate a transgenic progeny, wherein the progeny comprises at least one heterologous nucleic acid sequence encoding a heterologous polypeptide and wherein the heterologous polypeptide is expressed by the transgenic avian.
